SEQUENCE LISTING (7 sequences)

SEQ ID NO: 1 - protein, 112 aa, Homo sapiens
ALDAAYCFRNVQDNCCLRPLYIDFKRDLGWKWIHEPKGYNANFCAGACPYLWSSDTQHSKVLSLYNTINPEASASPCCVSQDLEPLTILYYIGKTPKIEQLSNMIVKSCKCS

SEQ ID NO: 2 - protein, 92 aa, synthetic construct
ALDAAYCFRNVQDNCCLRPLYIDFKRDLGWKWIHEPKGYNANFCAGACPYRASKSPSCVSQDLEPLTILYYIGKTPKIEQLSNMIVKSCKCS

SEQ ID NO: 3 - protein, 92 aa, synthetic construct
ALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 4 - protein, 92 aa, synthetic construct
ALDAAYCFRNVQDNCCLRPLYIDFRKDLGWKWIHEPKGYNANFCAGACPYRASKSPRCRSQDLEPLTIVYYVGRKPKVEQLSNMIVKSCKCS

SEQ ID NO: 5 - protein, 92 aa, synthetic construct
ALDAAYVFRNVQDNCALRPLYIDFRRDLGWKWIHEPKGYNANFCAGACPYRASKSPSCVSQDLEPLTILYYIGRTPKIEQLSNMIVKSCKCS

SEQ ID NO: 6 - protein, 92 aa, synthetic construct
ALDAAYVFRNVQDNCALRPLYIDFRKDLGWKWIHEPKGYNANFCAGACPYRASKSPRCRSQDLEPLTIVYYVGRKPKVEQLSNMIVKSCKCS

SEQ ID NO: 7 - protein, 115 aa, synthetic construct
KEVLASSQEALVVTERKYLKSDWCKLRPLYIDFRKDLGWKWIHEPKGYNANFCYGQCNSFYIPRHVKKEEDSFQSSAFCVSQDLEPLTIVYYVGRKPKVEQLSNMIVKSCRCMSV

(19) United States
(12) Patent Application Publication
(10) Pub. No.: US 2024/0425559 A1
(43) Pub. Date: Dec. 26, 2024

(54) ENGINEERED TGF-BETA MONOMERS AND METHODS OF USE

(71) Applicant: University of Pittsburgh - Of the Commonwealth System of Higher Education, Pittsburgh, PA (US)

(72) Inventors: Andrew P. Hinck, Pittsburgh, PA (US); Kristin DePeaux, Pittsburgh, PA (US); Greg M. Delgoffe, Pittsburgh, PA (US)

(73) Assignee: University of Pittsburgh - Of the Commonwealth System of Higher Education, Pittsburgh, PA (US)

(21) Appl. No.: 18/700,446

(22) PCT Filed: Oct. 11, 2022
(86) PCT No.: PCT/US2022/077879

§ 371 Date: Apr. 11, 2024

Related U.S. Application Data
(60) Provisional application No. 63/254,249, filed on Oct. 11, 2021.

(51) Int. Cl. C07K 14/495; A61K 38/00

(52) U.S. Cl. CPC C07K 14/495; A61K 38/00; C07K 2319/30

(57) ABSTRACT

Recombinant TGF-β2 monomers engineered to prevent dimerization and block TGF-β signaling are described. The engineered monomers lack the ability to bind and recruit TGF-β type I receptor (TbRI), but retain the capacity to bind the high affinity TGF-β type II receptor (TbRII). The TGF-β2 monomers also include additional modifications that increase their affinity for TbRII, reduce their aggregation and/or improve their folding. Nucleic acid molecules and vectors encoding the recombinant TGF-β2 monomers are also described. Isolated cells, such as T cells, can be re-programmed with a TGF-β2 monomer-encoding nucleic acid or vector to secrete the monomer. Use of the recombinant TGF-β2 monomers and/or cells producing the recombinant TGF-β2 monomers, to inhibit TGF-β signaling, such as to treat disorders associated with aberrant TGF-β signaling, are also described.

### CROSS REFERENCE TO RELATED APPLICATIONS

This application claims the benefit of U.S. Provisional Application No. 63/254,249, filed Oct. 11, 2021, which is herein incorporated by reference in its entirety.

### ACKNOWLEDGMENT OF GOVERNMENT SUPPORT

This invention was made with government support under grant number CA247129 awarded by the National Institutes of Health. The government has certain rights in the invention.

### FIELD

This disclosure concerns transforming growth factor (TGF)-β monomers with improved properties, and their use for inhibiting TGF-β signaling and treating disorders associated with aberrant TGF-β signaling.

### BACKGROUND

TGF-β is an important target for cancer immunotherapy because TGF-β-mediated immunosuppression causes a cytotoxic-deficient environment, with T regulatory (T_{reg}) cells suppressing anti-tumor immunity through the secretion of immunosuppressive cytokines and the direct inhibition of effector T-cells. Overexpression of TGF-β in the tumor microenvironment (TME) has been correlated with a high tumor burden and poor clinical outcomes. Furthermore, checkpoint therapy non-responder patients have a T-cell depleted phenotype due to TGF-β mediated immune exclusion. TGF-β inhibitors, as adjuncts to PD-1 and PD-L1 therapy, have been further shown to surpass checkpoint monotherapy in vivo and such approaches are being pursued in clinical trials. TGF-β isoforms also potently stimulate the accumulation of matrix proteins, such as collagen and fibronectin, and can drive fibrotic disorders such as idiopathic pulmonary fibrosis (IPF), renal fibrosis, cardiac fibrosis, and coronary restenosis. IPF, which is characterized by a progressive loss of lung function that occurs in older adults, has a mean survival rate of 5 years after diagnosis. Though the mechanisms triggering various forms of fibrosis are diverse, they share a common induction of increased levels of TGF-β proteins. In IPF and renal fibrosis, increased levels of the TGF-β proteins stimulate the activation and differentiation of fibroblasts into myofibroblasts, which cause aberrant deposition of extracellular matrix (ECM), leading to scarring and reduced organ function.

Inhibition of TGF-β in the context of both cancer and fibrosis using neutralizing antibodies has limited efficacy, likely because the majority of TGF-β is stored as a latent protein in the ECM, making it inaccessible to inhibition. TGF-β receptor kinase inhibitors have greater accessibility to their target, the kinase domains of the TGF-β type I and type II receptors, but lack specificity and inhibit not only other TGF-β family type I receptors, but also non-TGF-β receptor kinases. Kinase inhibitors have not progressed past phase II in clinical trials and currently there are no FDA-approved TGF-β inhibitors. Thus, a need exists for improved therapies for treating disorders associated with aberrant TGF-β signaling.

### SUMMARY

Described herein are TGF-β2 monomers engineered to prevent dimerization and block TGF-β signaling. The engineered monomers lack the ability to bind and recruit TGF-β type I receptor (TβRI), but retain the capacity to bind the high affinity TGF-β type II receptor (TβRII). The disclosed TGF-β2 monomers also include additional modifications that increase their affinity for TβRII, reduce their aggregation and/or improve their folding. The disclosed TGF-β2 monomers and compositions thereof can be used, for example, to treat disorders associated with aberrant TGF-β signaling, for example fibrotic disorders and cancer.

Provided herein are recombinant TGF-β2 monomers that include a deletion of the α3 (heel) helix corresponding to amino acid residues 52-71 of wild-type human TGF-β2 (set forth as SEQ ID NO: 1), and a cysteine to arginine or serine substitution at an amino acid residue corresponding to residue 77 of SEQ ID NO: 1; these modifications prevent dimerization of the monomers. The TGF-β2 monomers further include a leucine to arginine substitution at an amino acid residue corresponding to residue 51 of SEQ ID NO: 1, and an alanine to lysine substitution at an amino acid residue corresponding to residue 74 of SEQ ID NO: 1; these modifications increase the net charge of the monomers. The TGF-β2 monomers also include a lysine to arginine substitution at an amino acid residue corresponding to residue 25 of SEQ ID NO: 1, and a lysine to arginine substitution at an amino acid residue corresponding to residue 94 of SEQ ID NO: 1, which increase the affinity of the monomers for TβRII. In some implementations, the TGF-β2 monomers further include one or more additional modifications that increase affinity of the monomers for TβRII, reduce aggregation and/or improve folding.

Also provided herein are engineered TGF-β2 monomers that are modified to include the cystine-knot region of protein related to Dan and Cerubus (PRDC), which enhances folding of the monomers.

Fusion proteins that include a TGF-β2 monomer and a heterologous protein are also provided. In some implementations, the heterologous protein includes a protein tag, an Fc domain, albumin, an albumin-binding polypeptide, an antibody, an antigen-binding fragment of an antibody or a targeting moiety.

Also provided are nucleic acid molecules and vectors that encode a recombinant TGF-β2 monomer or fusion protein disclosed herein. Further provided are isolated cells, such as isolated T cells, that include the recombinant TGF-β2 monomer- or fusion protein-encoding nucleic acid molecule or vector.

Further provided are compositions that include a recombinant TGF-β2 monomer, fusion protein, nucleic acid molecule, vector or isolated cell disclosed herein and a pharmaceutically acceptable carrier, diluent, or excipient.

Also provided are methods of inhibiting TGF-β signaling in a cell by contacting the cell with a recombinant TGF-β2 monomer, fusion protein, nucleic acid molecule, vector, or composition disclosed herein.

Further provided are methods of inhibiting TGF-β signaling in a subject having a disease or disorder associated with aberrant TGF-β signaling. In some implementations, the method includes administering to the subject an effective amount of a recombinant TGF-β2 monomer, fusion protein, nucleic acid molecule, vector, isolated cell (such as a T cell) or composition disclosed herein. Methods of treating a disease or disorder associated with aberrant TGF-β signaling in a subject are further provided. In some implementations, the method includes administering to the subject a therapeutically effective amount of a recombinant TGF-β2 monomer, fusion protein, nucleic acid molecule, vector, isolated cell (such as a T cell) or composition disclosed herein. In some examples of the disclosed methods, the disease or disorder associated with aberrant TGF-β signaling is a fibrotic disorder, a cancer, an ocular disorder or a genetic disorder of connective tissue.

The foregoing and other objects and features of the disclosure will become more apparent from the following detailed description, which proceeds with reference to the accompanying figures.

### BRIEF DESCRIPTION OF THE DRAWINGS

FIGS. 1A-1E: Sequence comparison of engineered TGF-β2 monomer mmTGF-β2-7M (SEQ ID NO: 3) to TGF-β2 (SEQ ID NO: 1) (FIG. 1A), mmTGF-β2-7M2R (SEQ ID NO: 4) (FIG. 1B), mmTGF-β2-2M-Del7_16 (SEQ ID NO: 5) (FIG. 1C), mmTGF-β2-7M-PRDC (SEQ ID NO: 7) (FIG. 1D), and mmTGF-β2-7M2R-Del7-16 (SEQ ID NO: 6) (FIG. 1E). Sequence differences are indicated by numerals under the two aligned sequences, with the identity of the numeral indicating the nature of the difference. Sequence identities are indicated by an asterisk. Shown below the sequences in FIG. 1A is the structure of the TGF-β3-(TβRII)₂-(TβRI)₂ complex (PDB 2PJY) (left) and the mmTGF-β2-7M-TβRII complex (PDB 5TX4) (right), with some of the main structural features highlighted.

FIGS. 2A-2F: Amide ¹H-¹⁵N one-bond shift correlation nuclear magnetic resonance (NMR) spectra of mmTGF-β2-7M2R (FIGS. 2A-2C) compared to the parent protein, mmTGF-β2-7M (FIGS. 2D-2F). Spectra were recorded at 37° C. in 10 mM phosphate buffer at pH 4.6 (FIGS. 2A and 2D) or pH 7.2, either in the absence of CHAPS in the buffer (FIGS. 2B and 2E) or with CHAPS added to a final concentration of 10 mM (FIGS. 2C and 2F).

FIGS. 3A-3C: Amide ¹H-¹⁵N one-bond shift correlation NMR spectra of mmTGF-β2-2M-Del7-16. Spectra were recorded at 37° C. in 10 mM phosphate buffer at pH 6.0 (FIG. 3B), or pH 4.5, either in the absence of CHAPS in the buffer (FIG. 3A) or with CHAPS added to a final concentration of 10 mM (FIG. 3C).

FIGS. 4A-4D: Amide ¹H-¹⁵N one-bond shift correlation NMR spectra of mmTGF-β2-7M-PRDC (FIGS. 4A-4C) and binding to TβRII as detected by native gel electrophoresis (FIG. 4D). Spectra were recorded at 37° C. in 10 mM phosphate buffer at pH 4.8 (FIG. 4A) or pH 6.0 (FIGS. 4B-4C). FIG. 4B and FIG. 4C differ only in the contour level at which the signals are plotted (FIG. 4B is plotted at a contour level closer to the noise compared to panel FIG. 4C). Native gel shown in FIG. 7 D was performed by running either 2 μg of TβRII alone (left most lane) or with the engineered TGF-β monomers added in the specified molar ratio (+A and +B indicate TβRII:engineered TGF-β monomer in either a 1:1 or 2:1 molar ratio, respectively).

FIG. 5: Binding of the engineered TGF-beta monomers (mmTGF-β2-7M, left; mmTGF-β2-7M2R, middle; and mmTGF-β2-2M-Del7-16, right) to the TGF-β type II receptor (TβRII) as detected by isothermal titration calorimetry (ITC). Upper panels depict the raw thermograms for three replicate titrations, while the lower panels depict the integrated heat (data points) for the three replicate titrations globally fit to a 1:1 binding isotherm (smooth line). Fitted parameters are provided in the Table at the bottom.

FIGS. 6A-6D: HEK-293 cell-based CAGA-Luc TGF-β reporter assay to assess inhibitory potency of the engineered TGF-beta monomers relative to one another. HEK-293 cells stably transfected with the TGF-β CAGA-Luc reporter were treated with the indicated engineered TGF-beta monomers at the concentrations specified for 30 minutes and then stimulated by the addition of 10 pM TGF-β3. Cells were harvested after 14 hours and assayed for luciferase activity. (FIG. 6A) mmTGF-β2-7M (SEQ ID NO: 3), IC₅₀ of 58.23 nM. (FIG. 6B) mmTGF-β2-7M2R (SEQ ID NO: 4), IC₅₀ of 53.29 nM. (FIG. 6C) mmTGF-β2-2M-Del7-16 (SEQ ID NO: 5), IC₅₀ of 111.0 nM. (FIG. 6D) mmTGF-β2-7M-PRDC (SEQ ID NO: 7), IC₅₀ of 282.5 nM. Data points and error bars shown correspond to the mean and standard deviation of triplicate measurements. Smooth curve corresponds to the fit to a standard dose response inhibition isotherm. Fitted IC₅₀ values are shown.

### SEQUENCE LISTING

The Sequence Listing is submitted as an ST.26 Sequence Listing XML file, named 8123-107062-03, created on Oct. 3, 2022, having a size of 7561 bytes, which is incorporated by reference herein. In the accompanying sequence listing:
SEQ ID NO: 1 is the amino acid sequence of wild-type human TGF-β2.
SEQ ID NO: 2 is the amino acid sequence of an engineered human TGF-β2 monomer designated mmTGF-β2.
SEQ ID NO: 3 is the amino acid sequence of an engineered human TGF-β2 monomer designated mmTGF-β2-7M.
SEQ ID NO: 4 is the amino acid sequence of an engineered human TGF-β2 monomer designated mmTGF-β2-7M2R.
SEQ ID NO: 5 is the amino acid sequence of an engineered human TGF-β2 monomer designated mmTGF-β2-2M-Del7-16.
SEQ ID NO: 6 is the amino acid sequence of an engineered human TGF-β2 monomer designated mmTGF-β2-7M2R-Del7-16.
SEQ ID NO: 7 is the amino acid sequence of an engineered human TGF-β2 monomer designated mmTGF-β2-7M-PRDC.

### DETAILED DESCRIPTION

### I. Abbreviations

CKGF cystine knot growth factor fold
ECM extracellular matrix
ER endoplasmic reticulum
GFD growth factor domain
IPF idiopathic pulmonary fibrosis
ITC isothermal titration calorimetry
NGF nerve growth factor
NMR nuclear magnetic resonance
PDGF platelet-derived growth factor
PRDC protein related to Dan and Cerubus
TGF-β transforming growth factor β
TβRI transforming growth factor-β type I receptor
TβRII transforming growth factor-β type II receptor
TME tumor microenvironment
VEGF vascular-endothelial growth factor

### II. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various implementations, the following explanations of terms are provided:

Aberrant (TGF-βsignaling): Abnormal or dysregulated TGF-β signaling. In the context of the present disclosure, “aberrant TGF-β signaling” refers to excessive (pathological) activation of the TGF-β signaling pathway.

Administration: To provide or give a subject an agent, such as a therapeutic agent (e.g. a TGF-β monomer), by any effective route. Exemplary routes of administration include, but are not limited to, injection or infusion (such as intratumoral, subcutaneous, intramuscular, intradermal, intraperitoneal, intrathecal, intravenous, intraprostatic, intracerebroventricular, intrastriatal, intracranial and into the spinal cord), oral, intraductal, sublingual, rectal, transdermal, intranasal, vaginal and inhalation routes.

Contacting: Placement in direct physical association; includes both in solid and liquid form. When used in the context of an in vivo method, “contacting” also includes administering.

Fibrosis: The formation of excess fibrous connective tissue in an organ or tissue in a reparative or reactive process. Fibrosis can occur in many different tissues of the body (such as heart, lung and liver), typically as the result of inflammation or damage. Fibrotic disorders include, but are not limited to, pulmonary fibrosis, cystic fibrosis, idiopathic pulmonary fibrosis, interstitial lung disease, liver cirrhosis, kidney fibrosis (such as from damage caused by diabetes), atrial fibrosis, endomyocardial fibrosis, atherosclerosis, restenosis and scleroderma. Fibrosis can also occur as a result of surgical complications, chemotherapeutic drugs, radiation, injury or burns.

Fusion protein: A protein comprising at least a portion of two different (heterologous) proteins. In some implementations herein, the fusion protein includes a TGF-β2 monomer fused to a protein tag, an Fc domain (such as a human Fc domain) or albumin.

Glycosylation: The process of covalent attachment of carbohydrate moieties to an asparagine (N-glycosylation), or serine or threonine residue (O-glycosylation). The level and type of glycosylation can vary in different host organisms used for recombinant expression. Novel glycosylation sites can be sequence engineered by introducing glycosylation sequons in solvent exposed regions of the protein. For example, the N-glycosylation sequon NX[S/T] can be introduced at one or more places within the sequence of certain implementations disclosed herein. Varying the type and extent of glycosylation has practical applications in modulating solubility, function and half-life, as well as enabling site-specific chemical conjugation.

Heterologous: Originating from a separate genetic source or species.

Isolated: An “isolated” biological component, such as a nucleic acid, protein (including antibodies), organelle, or recombinant virus, has been substantially separated or purified away from other biological components in the environment (such as a cell) in which the component occurs, such as other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids or proteins. Isolated does not require absolute purity, and can include protein, peptide, nucleic acid molecules or viruses that are at least 50% isolated, such as at least 75%, 80%, 90%, 95%, 98%, 99%, or even 99.9% isolated.

Modification: A change in the sequence of a nucleic acid or protein sequence. For example, amino acid sequence modifications include, for example, substitutions, insertions and deletions, or combinations thereof. Insertions include amino and/or carboxyl terminal fusions as well as intrasequence insertions of single or multiple amino acid residues. Deletions are characterized by the removal of one or more amino acid residues from the protein sequence. In some implementations herein, the modification (such as a substitution, insertion or deletion) results in a change in function, such as a reduction or enhancement of a particular activity of a protein (for example, reducing aggregation, improved folding or increase affinity for a target protein). Substitutional modifications are those in which at least one residue has been removed and a different residue inserted in its place. Amino acid substitutions are typically of single residues, but can occur at a number of different locations at once. Substitutions, deletions, insertions or any combination thereof may be combined to arrive at a final mutant sequence. These modifications can be prepared by modification of nucleotides in the DNA encoding the protein, thereby producing DNA encoding the modification. Techniques for making insertion, deletion and substitution mutations at predetermined sites in DNA having a known sequence are known. A “modified” protein or nucleic acid is one that has one or more modifications as outlined above.

Monomer: A single molecular unit (such as a protein) that is capable of binding to other molecular units to form dimers or polymers. In the context of the present disclosure, a “TGF-β2 monomer” is a single TGF-β2 polypeptide chain, the wild-type version of which can bind other TGF-β2 monomers to form dimers. In some implementations herein, the recombinant TGF-β2 monomers have been engineered to prevent dimerization. In other implementations herein, the recombinant TGF-β2 monomers which have been engineered to prevent their direct dimerization can be fused to heterologous proteins that are themselves capable of dimerization (e.g., an Fc domain of an IgG).

Neoplasia, malignancy, cancer or tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma).

PEGylation: The process of both covalent and non-covalent attachment or amalgamation of polyethylene glycol (PEG) polymer chains to molecules and macrostructures, such as a drug, therapeutic protein or vesicle, which is then referred to as PEGylated (or pegylated). PEGylation is routinely achieved by incubation of a reactive derivative of PEG with the target molecule. The covalent attachment of PEG to a drug or therapeutic protein can mask the agent from the host's immune system (reduced immunogenicity and antigenicity), and increase the hydrodynamic size (size in solution) of the agent, which prolongs its circulatory time by reducing renal clearance. PEGylation can also provide water solubility to hydrophobic drugs and proteins.

Peptide or Polypeptide: A polymer in which the monomers are amino acid residues which are joined together through amide bonds. When the amino acids are alpha-amino acids, either the L-optical isomer or the D-optical isomer can be used. The terms “peptide,” “polypeptide” or “protein” as used herein are intended to encompass any amino acid sequence and include modified sequences. The terms “peptide” and “polypeptide” are specifically intended to cover naturally occurring proteins, as well as those which are recombinantly or synthetically produced.

Conservative amino acid substitutions are those substitutions that, when made, least interfere with the properties of the original protein, that is, the structure and especially the function of the protein is conserved and not significantly changed by such substitutions. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamine or aspartic acid; or (d) a residue having a bulky side chain, for example, phenylalanine, is substituted for (or by) one not having a side chain, for example, glycine.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds, molecules or agents (e.g., a recombinant TGF-β2 monomer).

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden (such as decrease in the volume or size of a tumor) or a decrease in the number of size of metastases. “Ameliorating” refers to the reduction in the number or severity of signs or symptoms of a disease.

Recombinant: A recombinant nucleic acid, protein or virus is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques. The term recombinant includes nucleic acids, proteins and viruses that have been altered by addition, substitution, or deletion of a portion of a natural nucleic acid molecule or protein.

Sequence identity/similarity: The identity between two or more nucleic acid sequences, or two or more amino acid sequences, is expressed in terms of the identity or similarity between the sequences. Sequence identity can be measured in terms of percentage identity; the higher the percentage, the more identical the sequences. Sequence similarity can be measured in terms of percentage similarity (which takes into account conservative amino acid substitutions); the higher the percentage, the more similar the sequences. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Subject: Living multi-cellular organisms, including vertebrate organisms, a category that includes both human and non-human mammals.

Tag: A molecule that can be attached to a protein or nucleic acid, such as for labeling, detection or purification purposes. In some implementations, the tag is a protein tag. In some implementations, the protein tag is an affinity tag (for example, Avitag, hexahistidine, chitin binding protein, maltose binding protein, or glutathione-S-transferase), an epitope tag (for example, V5, c-myc, HA or FLAG) or a fluorescent tag (e.g., GFP or another well-known fluorescent protein).

Therapeutically effective amount: A quantity of a compound or composition, for instance, a recombinant TGF-β2 monomer, sufficient to achieve a desired effect in a subject being treated. For instance, this can be the amount necessary to inhibit or block TGF-β signaling in a cell. In other instances, this can be the amount necessary to inhibit or suppress growth of a tumor. In one implementation, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment. In one implementation, a therapeutically effective amount is the amount necessary to increase the survival time of a subject such as by at least 3 months, at least 4 months, at least 5 months, at least 6 months, at least 9 months, at least 1 year, at least 1.5 years, at least 2 years, at least 3 years, at least 4 years, or at least 5 years, for example as compared to a survival time of a subject with the same cancer without the treatment with the recombinant TGF-β2 monomer. In other instances, the therapeutically effective amount is an effect necessary to inhibit or reduce fibrosis, such as reduce by at least 10%, at least 20%, at least 50%, at least 75%, at least 80%, at least 90%, at least 95%, or even 100%, compared to prior to treatment. When administered to a subject, a dosage will generally be used that will achieve target tissue concentrations (for example, in tumors) that have been shown to achieve a desired in vitro effect.

Transforming growth factor-β (TGF-β): A secreted, multi-functional protein that regulates proliferation, cellular differentiation and a number of other cellular functions. Many cells synthesize TGF-β and nearly all cells express receptors for TGF-β. The term “TGF-β” refers to three different protein isoforms, TGF-β1, TGF-β2 and TGF-β3, encoded by the genes TGFB1, TGFB2, TGFB3, respectively.

TGF-β signaling pathway: A signaling pathway involved in a number of cellular processes, such as cell proliferation, differentiation and apoptosis. Members of the TGF-β pathway include, but are not limited to, TGF-β1, TGF-β2, TGF-β3, TGF-β receptor type I and TGF-β receptor type II.

TGF-β receptor: The term “TGF-β receptor” includes TGF-β receptor type I (TβRI, encoded by TGFBR1) and TGF-β receptor type II (TβRII, encoded by TGFBR2). TGF-β receptors are serine/threonine protein kinases. The type I and type II TGF-β receptors form a heterodimeric complex when bound to TGF-β, transducing the TGF-β signal from the cell surface to the cytoplasm.

### III. Recombinant TGF-β2 Monomers

Disclosed herein are TGF-β2 monomers engineered to prevent dimerization and block TGF-β signaling. The engineered monomers lack the ability to bind and recruit TGF-β type I receptor (TβRI), but retain the capacity to bind the high affinity TGF-β type II receptor (TβRII). The disclosed TGF-β2 monomers also include additional modifications that increase their affinity for TβRII, reduce their aggregation and/or improve their folding. The disclosed TGF-β2 monomers and compositions thereof can be used, for example, to inhibit TGF-β signaling in a cell or subject, or to treat disorders associated with aberrant TGF-β signaling, for example fibrotic disorders, cancer, ocular diseases or genetic disorders of connective tissue.

Provided herein are recombinant TGF-β2 monomers that include a deletion of the α3 helix corresponding to amino acid residues 52-71 of wild-type human TGF-β2 (set forth as SEQ ID NO: 1), and a cysteine to arginine (or serine) substitution at an amino acid residue corresponding to residue 77 of SEQ ID NO: 1; these modifications prevent dimerization of the monomers. The TGF-β2 monomers further include a leucine to arginine substitution at an amino acid residue corresponding to residue 51 of SEQ ID NO: 1, and an alanine to lysine substitution at an amino acid residue corresponding to residue 74 of SEQ ID NO: 1; these modifications increase the net charge of the monomers. The TGF-β2 monomers also include a lysine to arginine substitution at an amino acid residue corresponding to residue 25 of SEQ ID NO: 1, and a lysine to arginine substitution at an amino acid residue corresponding to residue 94 of SEQ ID NO: 1, which enhance the affinity for TβRII. The TGF-β2 monomers optionally further include one or more additional modifications that increase affinity of the monomers for TβRII, reduce aggregation and/or improve folding.

In some implementations, the TGF-β2 monomer further includes an arginine to lysine substitution at an amino acid residue corresponding to residue 26 of SEQ ID NO: 1; a valine to arginine substitution at an amino acid residue corresponding to residue 79 of SEQ ID NO: 1; a leucine to valine substitution at an amino acid residue corresponding to residue 89 of SEQ ID NO: 1; an isoleucine to valine substitution at an amino acid residue corresponding to residue 92 of SEQ ID NO: 1; a threonine to lysine substitution at an amino acid residue corresponding to residue 95 of SEQ ID NO: 1; and an isoleucine to valine substitution at an amino acid residue corresponding to residue 98 of SEQ ID NO: 1. In some examples, the TGF-β2 monomer has a cysteine to arginine substitution at an amino acid residue corresponding to residue 77 of SEQ ID NO: 1. In particular examples, the amino acid sequence of the TGF-β2 monomer is at least 85%, at least 90%, at least 95%, at least 96%, at least 97%, at least 98% or at least 99% identical to SEQ ID NO: 4 (while retaining the above-listed amino acid substitutions). In specific non-limiting examples, the amino acid sequence of the TGF-β2 monomer comprises or consists of the amino acid sequence of mmTGF-β2-7M2R set forth herein as SEQ ID NO: 4.

In other implementations, the TGF-β2 monomer further includes a cysteine to valine substitution at an amino acid residue corresponding to residue 7 of SEQ ID NO: 1; and a cysteine to alanine substitution at an amino acid residue corresponding to residue 16 of SEQ ID NO: 1. In some examples, the TGF-β2 monomer has a cysteine to serine substitution at an amino acid residue corresponding to residue 77 of SEQ ID NO: 1. In particular examples, the amino acid sequence of the TGF-β2 monomer is at least 85%, at least 90%, at least 95%, at least 96%, at least 97%, at least 98% or at least 99% identical to SEQ ID NO: 5 (while retaining the above-listed amino acid substitutions). In specific non-limiting examples, the amino acid sequence of the TGF-β2 monomer comprises or consists of the amino acid sequence of mmTGF-β2-2M-Del7-16 set forth herein as SEQ ID NO: 5.

In other implementations, the TGF-β2 monomer further includes a cysteine to valine substitution at an amino acid residue corresponding to residue 7 of SEQ ID NO: 1; a cysteine to alanine substitution at an amino acid residue corresponding to residue 16 of SEQ ID NO: 1; an arginine to lysine substitution at an amino acid residue corresponding to residue 26 of SEQ ID NO: 1; a valine to arginine substitution at an amino acid residue corresponding to residue 79 of SEQ ID NO: 1; a leucine to valine substitution at an amino acid residue corresponding to residue 89 of SEQ ID NO: 1; an isoleucine to valine substitution at an amino acid residue corresponding to residue 92 of SEQ ID NO: 1; a threonine to lysine substitution at an amino acid residue corresponding to residue 95 of SEQ ID NO: 1; and an isoleucine to valine substitution at an amino acid residue corresponding to residue 98 of SEQ ID NO: 1. In some examples, the TGF-β2 monomer has a cysteine to arginine substitution at an amino acid residue corresponding to residue 77 of SEQ ID NO: 1. In particular examples, the amino acid sequence of the TGF-β2 monomer is at least 85%, at least 90%, at least 95%, at least 96%, at least 97%, at least 98% or at least 99% identical to SEQ ID NO: 6 (while retaining the above-listed amino acid substitutions). In specific non-limiting examples, the amino acid sequence of the TGF-β2 monomer comprises or consists of the amino acid sequence of mmTGF-β2-7M2R-Del7-16 (“variant 1” or “var1”) set forth herein as SEQ ID NO: 6.

Also provided herein are recombinant TGF-β2 monomers that are modified to include the cystine-knot region of protein related to Dan and Cerubus (PRDC) to enhance folding of the monomers. In some implementations, the amino acid sequence of the TGF-β2 monomer is at least 85%, at least 90%, at least 95%, at least 96%, at least 97%, at least 98% or at least 99% identical to SEQ ID NO: 7. In particular examples, the amino acid sequence of the TGF-β2 monomer comprises or consists of the amino acid sequence of mmTGF-β2-7M-PRDC set forth herein as SEQ ID NO: 7.

In some implementations herein, the recombinant TGF-β2 monomer is PEGylated, glycosylated, hyper-glycosylated, or includes another modification that prolongs circulatory time.

In some implementations, the recombinant TGF-β2 monomer further includes a radiotherapy agent, a cytotoxic agent for chemotherapy, a drug, an imaging agent, a fluorescent dye, or a fluorescent protein tag.

Also provided herein are fusion proteins that include a TGF-β2 monomer and a heterologous protein. In some implementations, the heterologous protein is a protein tag. In some examples, the protein tag is an affinity tag (for example, Avitag, hexahistidine, chitin binding protein, maltose binding protein, or glutathione-S-transferase), an epitope tag (for example, V5, c-myc, HA or FLAG) or a fluorescent tag (e.g., GFP or another well-known fluorescent protein).

In other implementations, the heterologous protein includes an Fc domain, such as a mouse or human Fc domain. In specific implementations, the heterologous protein promotes intermolecular association into homodimeric (for example, Fc domain from human IgG1, IgG2, IgG3), heterodimeric (for example, an engineered Fc domain, E/K coiled-coil), or multimeric (for example, pentabodies, nanoparticles) states of the fusion protein. Thus, in some examples, the fusion protein is a single-chain polypeptide. In other examples, the fusion protein forms a dimeric or multimeric polypeptide. In specific examples, the fusion protein is heterodimeric.

In other implementations, the heterologous protein is albumin, an albumin-binding protein or agent, or another protein that increases circulatory time of the TGF-β monomer in vivo.

Further provided herein are isolated nucleic acid molecules that encode a recombinant TGF-β2 monomer or fusion protein disclosed herein. In some implementations, the nucleic acid molecule is operably linked to a promoter, such as a T cell specific promoter.

Also provided are vectors that include a disclosed nucleic acid molecule. In some examples, the vector is a viral vector, such as a lentiviral vector. Further provided are isolated cells that include a nucleic acid molecule or vector disclosed herein. In some examples, the cell is a T cell. The cells can be autologous to the subject, or they can be heterologous (allogeneic).

Further provided herein are compositions that include a recombinant TGF-β2 monomer, fusion protein, nucleic acid molecule, vector, or isolated cell disclosed herein, and a pharmaceutically acceptable carrier, diluent, or excipient.

Also provided herein are methods of inhibiting TGF-β signaling in a cell. In some implementations, the method includes contacting the cell with an effective amount of a recombinant TGF-β2 monomer, fusion protein, nucleic acid molecule, vector, isolated cell or composition disclosed herein. In some examples, the method is an in vitro method. In other examples, the method is an ex vivo. In yet other examples, the method is an in vivo method.

Further provided are methods of inhibiting TGF-β signaling in a subject having a disease or disorder associated with aberrant TGF-β signaling. In some implementations, the method includes administering to the subject an effective amount of a recombinant TGF-β2 monomer, fusion protein, nucleic acid molecule, vector, isolated cell or composition disclosed herein. In some examples, the disease or disorder associated with aberrant TGF-β signaling is a fibrotic disorder, such as but not limited to, pulmonary fibrosis, cystic fibrosis, idiopathic pulmonary fibrosis, interstitial lung disease, liver cirrhosis, kidney fibrosis (such as from damage caused by diabetes), atrial fibrosis, endomyocardial fibrosis, atherosclerosis, restenosis, scleroderma, or fibrosis caused by a surgical complication, chemotherapeutic drugs, radiation, injury or burns. In other examples, the disease or disorder associated with aberrant TGF-β signaling is breast cancer, brain cancer, pancreatic cancer, prostate cancer, skin cancer, bladder cancer, liver cancer, ovarian cancer, renal cancer, endometrial cancer, colorectal cancer, gastric cancer, skin cancer (such as malignant melanoma), or thyroid cancer. In other examples, the disease or disorder associated with aberrant TGF-β signaling is an ocular disease. In yet other examples, the disease or disorder associated with aberrant TGF-β signaling is a genetic disorder of connective tissue.

Methods of treating a disease or disorder associated with aberrant TGF-β signaling in a subject are further provided. In some implementations, the method includes administering to the subject a therapeutically effective amount of a recombinant TGF-β2 monomer, fusion protein, nucleic acid molecule, vector, isolated cell or composition disclosed herein. In some examples, the disease or disorder associated with aberrant TGF-β signaling is a fibrotic disorder, such as but not limited to, pulmonary fibrosis, cystic fibrosis, idiopathic pulmonary fibrosis, interstitial lung disease, liver cirrhosis, kidney fibrosis (such as from damage caused by diabetes), atrial fibrosis, endomyocardial fibrosis, atherosclerosis, restenosis, scleroderma, or fibrosis caused by a surgical complication, chemotherapeutic drugs, radiation, injury or burns. In other examples, the disease or disorder associated with aberrant TGF-β signaling is breast cancer, brain cancer, pancreatic cancer, prostate cancer, skin cancer, bladder cancer, liver cancer, ovarian cancer, renal cancer, endometrial cancer, colorectal cancer, gastric cancer, skin cancer (such as malignant melanoma), or thyroid cancer. In other examples, the disease or disorder associated with aberrant TGF-β signaling is an ocular disease. In yet other examples, the disease or disorder associated with aberrant TGF-β signaling is a genetic disorder of connective tissue.

### IV. Administration of Engineered TGF-β Monomers

Compositions, such as pharmaceutical compositions, that include a recombinant human TGF-β2 monomer, fusion protein, or nucleic acid molecule or vector encoding a TGF-β2 monomer or fusion protein, are provided herein. Also provided are compositions that include an isolated cell, such as a T cell, comprising a vector encoding a recombinant human TGF-β2 monomer (or fusion protein thereof). In some implementations, the composition includes a pharmaceutically acceptable carrier, diluent or excipient.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, PA, 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

With regard to administration of cells, a variety of aqueous carriers can be used, for example, buffered saline and the like, for introducing the cells. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration in these formulations can vary widely, and will be selected primarily based on fluid volumes, viscosities, body weight and the like in accordance with the particular mode of administration selected and the subject's needs.

The dosage form of the composition will be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, starch, or magnesium stearate. Actual methods of preparing such dosage forms are known, or will be apparent, to those skilled in the art.

The compositions, such as pharmaceutical compositions, that include a recombinant human TGF-β2 monomer or fusion protein (or nucleic acid molecule/vector encoding a TGF-β2 monomer or fusion protein), can be formulated in unit dosage form, suitable for individual administration of precise dosages. The amount of TGF-β2 monomer, fusion protein, nucleic acid molecule or vector administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

The TGF-β2 monomers, or compositions thereof, can be administered to humans or other animals on whose tissues they are effective in various manners such as topically, orally, intravenously, intramuscularly, intraperitoneally, intranasally, intradermally, intrathecally, subcutaneously, via inhalation or via suppository. The particular mode of administration and the dosage regimen will be selected by the attending clinician, taking into account the particulars of the case (e.g. the subject, the disease, the disease state involved, and whether the treatment is prophylactic). Treatment can involve daily or multi-daily doses of compound(s) over a period of a few days to months, or even years.

### V. Exemplary Clauses

Clause 1. A recombinant transforming growth factor (TGF)-β2 monomer, comprising:
a cysteine to serine substitution, or a cysteine to arginine substitution, at an amino acid residue corresponding to residue 77 of SEQ ID NO: 1;
a deletion of the α3 helix corresponding to amino acid residues 52-71 of SEQ ID NO: 1;
a lysine to arginine substitution at an amino acid residue corresponding to residue 25 of SEQ ID NO: 1;
a leucine to arginine substitution at an amino acid residue corresponding to residue 51 of SEQ ID NO: 1;
an alanine to lysine substitution at an amino acid residue corresponding to residue 74 of SEQ ID NO: 1;
a lysine to arginine substitution at an amino acid residue corresponding to residue 94 of SEQ ID NO: 1; and
   (i) an arginine to lysine substitution at an amino acid residue corresponding to residue 26 of SEQ ID NO: 1; a valine to arginine substitution at an amino acid residue corresponding to residue 79 of SEQ ID NO: 1; a leucine to valine substitution at an amino acid residue corresponding to residue 89 of SEQ ID NO: 1; an isoleucine to valine substitution at an amino acid residue corresponding to residue 92 of SEQ ID NO: 1; a threonine to lysine substitution at an amino acid residue corresponding to residue 95 of SEQ ID NO: 1; and an isoleucine to valine substitution at an amino acid residue corresponding to residue 98 of SEQ ID NO: 1;
   (ii) a cysteine to valine substitution at an amino acid residue corresponding to residue 7 of SEQ ID NO: 1; and a cysteine to alanine substitution at an amino acid residue corresponding to residue 16 of SEQ ID NO: 1; or
   (iii) a cysteine to valine substitution at an amino acid residue corresponding to residue 7 of SEQ ID NO: 1; a cysteine to alanine substitution at an amino acid residue corresponding to residue 16 of SEQ ID NO: 1; an arginine to lysine substitution at an amino acid residue corresponding to residue 26 of SEQ ID NO: 1; a valine to arginine substitution at an amino acid residue corresponding to residue 79 of SEQ ID NO: 1; a leucine to valine substitution at an amino acid residue corresponding to residue 89 of SEQ ID NO: 1; an isoleucine to valine substitution at an amino acid residue corresponding to residue 92 of SEQ ID NO: 1; a threonine to lysine substitution at an amino acid residue corresponding to residue 95 of SEQ ID NO: 1; and an isoleucine to valine substitution at an amino acid residue corresponding to residue 98 of SEQ ID NO: 1.

Clause 2. The recombinant TGF-β2 monomer of clause 1, comprising a cysteine to arginine substitution at an amino acid residue corresponding to residue 77 of SEQ ID NO: 1.

Clause 3. The recombinant TGF-β2 monomer of clause 2, wherein the amino acid sequence of the TGF-β2 monomer comprises or consists of SEQ ID NO: 4.

Clause 4. The recombinant TGF-β2 monomer of clause 2, wherein the amino acid sequence of the TGF-β2 monomer comprises or consists of SEQ ID NO: 6.

Clause 5. The recombinant TGF-β2 monomer of clause 1, comprising a cysteine to serine substitution at an amino acid residue corresponding to residue 77 of SEQ ID NO: 1.

Clause 6. The recombinant TGF-β2 monomer of clause 5, wherein the amino acid sequence of the TGF-β2 monomer comprises or consists of SEQ ID NO: 5.

Clause 7. A recombinant transforming growth factor (TGF)-β2 monomer, wherein the amino acid sequence of the TGF-β2 monomer comprises or consists of SEQ ID NO: 7.

Clause 8. The recombinant TGF-β2 monomer of any one of clauses 1-7, which is PEGylated.

Clause 9. The recombinant TGF-β2 monomer of any one of clauses 1-7, which is glycosylated or hyper-glycosylated.

Clause 10. The recombinant TGF-β2 monomer of any one of clauses 1-9, further comprising a radiotherapy agent, a cytotoxic agent for chemotherapy, a drug, an imaging agent, a fluorescent dye, or a fluorescent protein tag.

Clause 11. A fusion protein comprising the recombinant TGF-β2 monomer of any one of clauses 1-10 and a heterologous protein.

Clause 12. The fusion protein of clause 11, wherein the heterologous protein comprises a protein tag, an Fc domain, albumin, an albumin-binding polypeptide, an antibody, an antigen-binding fragment of an antibody or a targeting moiety.

Clause 13. The fusion protein of clause 11 or clause 12, wherein the fusion protein is a single-chain polypeptide.

Clause 14. The fusion protein of clause 11 or clause 12, wherein the fusion protein forms a dimeric polypeptide.

Clause 15. The fusion protein of clause 11 or clause 12, wherein the fusion protein is heterodimeric.

Clause 16. The fusion protein of clause 11 or clause 12, wherein the fusion protein is multimeric.

Clause 17. An isolated nucleic acid molecule encoding the recombinant TGF-β2 monomer of any one of clauses 1-10 or the fusion protein of any one of clauses 11-16.

Clause 18. The nucleic acid molecule of clause 17 operably linked to a promoter.

Clause 19. A vector comprising the nucleic acid molecule of clause 17 or clause 18.

Clause 20. An isolated cell comprising the nucleic acid molecule of clause 17 or clause 18, or the vector of clause 19.

Clause 21. The isolated cell of clause 20, wherein the cell is a T lymphocyte.

Clause 22. A composition comprising:
the recombinant TGF-β2 monomer of any one of clauses 1-10, the fusion protein of any one of clauses 11-16, the nucleic acid molecule of clause 17 or clause 18, the vector of clause 19, or the isolated cell of clause 20 or clause 21; and
a pharmaceutically acceptable carrier, diluent, or excipient.

Clause 23. A method of inhibiting TGF-β signaling in a cell, comprising contacting the cell with an effective amount of the recombinant TGF-β2 monomer of any one of clauses 1-10, the fusion protein of any one of clauses 11-16, the nucleic acid molecule of clause 17 or clause 18, the vector of clause 19, the isolated cell of clause 20 or clause 21, or the composition of clause 22.

Clause 24. A method of inhibiting TGF-β signaling in a subject having a disease or disorder associated with aberrant TGF-β signaling, comprising administering to the subject an effective amount of the recombinant TGF-β2 monomer of any one of clauses 1-10, the fusion protein of any one of clauses 11-16, the nucleic acid molecule of clause 17 or clause 18, the vector of clause 19, the isolated cell of clause 20 or clause 21, or the composition of clause 22.

Clause 25. A method of treating a disease or disorder associated with aberrant TGF-β signaling in a subject, comprising administering to the subject a therapeutically effective amount of the recombinant TGF-β2 monomer of any one of clauses 1-10, the fusion protein of any one of clauses 11-16, the nucleic acid molecule of clause 17 or clause 18, the vector of clause 19, the isolated cell of clause 20 or clause 21, or the composition of clause 22.

Clause 26. The method of clause 24 or clause 25, wherein the disease or disorder associated with aberrant TGF-β signaling is a fibrotic disorder.

Clause 27. The method of clause 24 or clause 25, wherein the disease or disorder associated with aberrant TGF-β signaling is breast cancer, brain cancer, pancreatic cancer, prostate cancer, skin cancer, bladder cancer, liver cancer, ovarian cancer, renal cancer, endometrial cancer, colorectal cancer, gastric cancer, skin cancer or thyroid cancer.

Clause 28. The method of clause 24 or clause 25, wherein the disease or disorder associated with aberrant TGF-β signaling is an ocular disease.

Clause 29. The method of clause 24 or clause 25, wherein the disease or disorder associated with aberrant TGF-β signaling is a genetic disorder of connective tissue.

### EXAMPLES

### Example 1: Modified TGF-β2 Monomers for In Vivo Administration

This example describes studies to evaluate modifications to enhance in vivo delivery of TGF-β2 monomers.

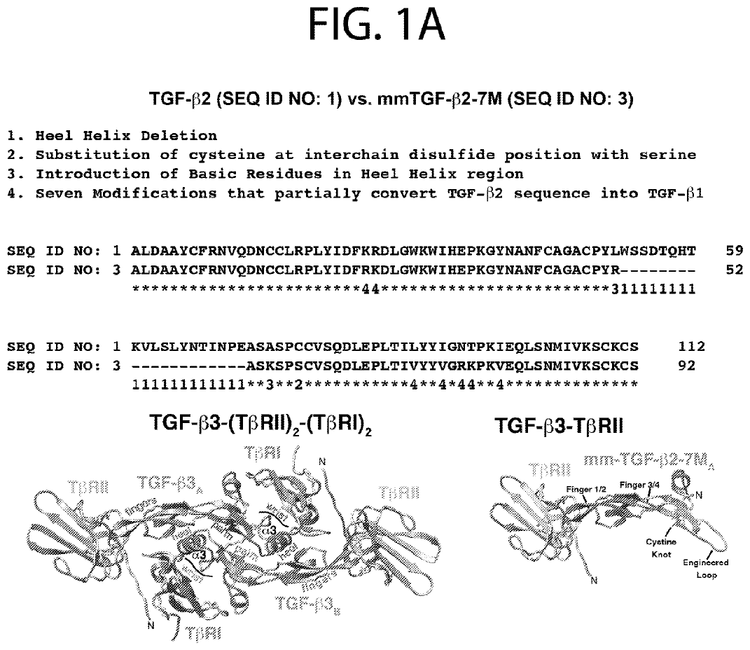

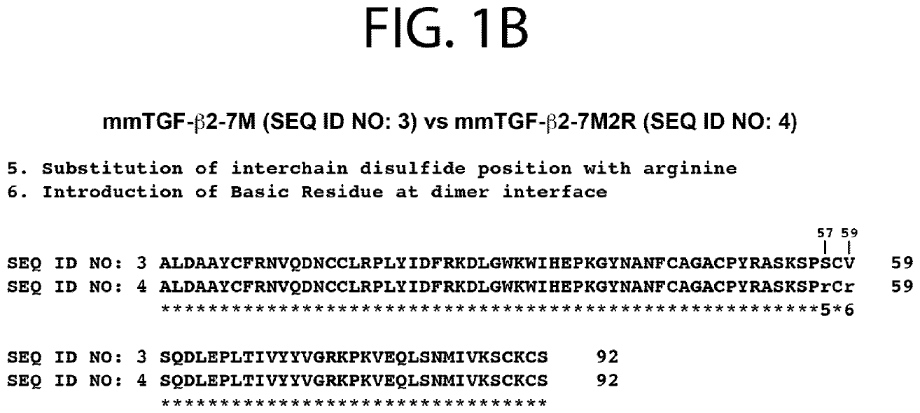

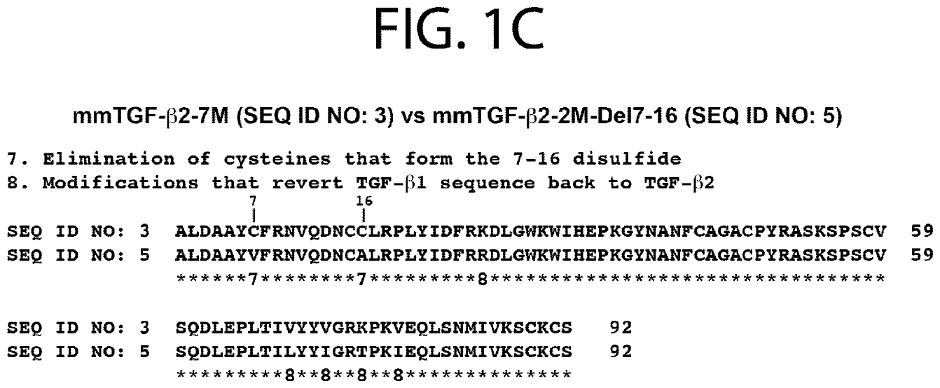

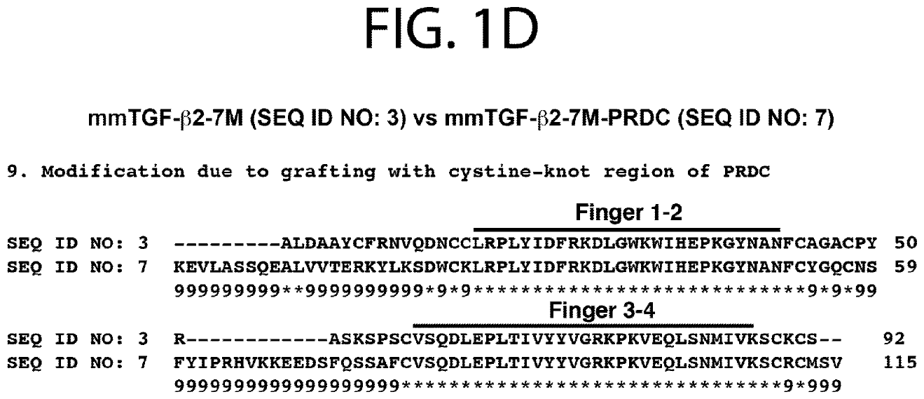

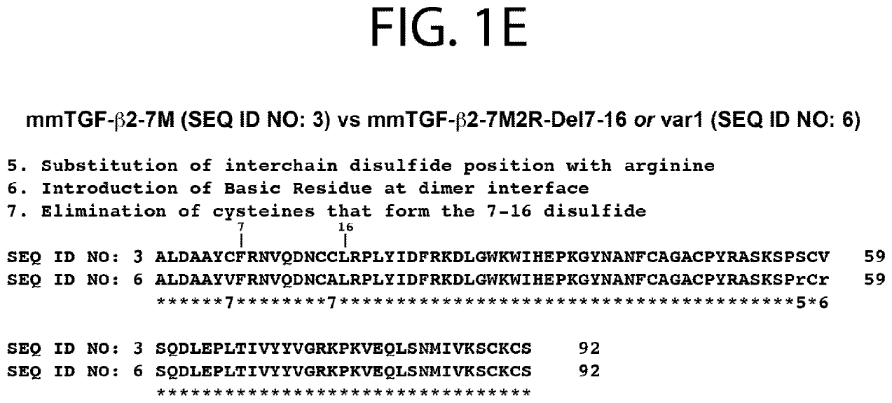

TGF-β monomers engineered to prevent dimerization, thereby preventing binding to and recruitment of TGF-β type I receptor (TβRI), are described in WO 2018/094173, which is herein incorporated by reference in its entirety. The TGF-β2 monomer mmTGF-β2-7M includes a C77S substitution and a deletion of the α3 helix to prevent dimerization, and further includes seven amino acid substitutions that enable high affinity TβRII binding and two substituted basic residues to increase its charge, and thus its solubility (FIG. 1A). To improve upon the properties of this monomer, four variants of mmTGF-β2-7M were generated (SEQ ID NOs: 4-7), which are described in Table 1 and FIGS. 1B-1E. The positions of the single amino acid substitutions and deletions are relative to human TGF-β2 set forth as SEQ ID NO: 1.

TABLE 1
TGF-β monomer variants

| | | | | Single | |
|---|---|---|---|---|---|
| Variant | SEQ ID | Variant | Length of | amino acid | |
| Name | NO | Description | monomer | substitutions | Deletion |
| mmTGF-β2 | 2 | Human TGF-β2 | 112 a.a. | C77S | None |
| | | monomer | | | |
| mmTGF-β2-7M | 3 | Human TGF-β2 | 92 a.a. | K25R, R26K, | Residues |
| | | monomer with α3 | | L51R, A74K, | 52-71 |
| | | replaced with a loop | | C77S, L89V, I92V, | |
| | | | | K94R T95K, I98V | |
| mmTGF-β2- | 4 | Human TGF-β2 mini- | 92 a.a. | K25R, R26K, | Residues |
| 7M2R | | monomer with | | L51R, A74K, | 52-71 |
| | | increased affinity for | | C77R, V79R, | |
| | | TβRII and reduced | | L89V, I92V, | |
| | | aggregation | | K94R, T95K, I98V | |
| mmTGF-β2- | 5 | Human TGF-β2 mini- | 92 a.a. | C7V, C16A, K25R, | Residues |
| 2M-Del7-16 | | monomer with | | L51R, A74K, | 52-71 |
| | | increased affinity for | | C77S, K94R | |
| | | TβRII and improved | | | |
| | | folding | | | |
| mmTGF-β2- | 6 | Human TGF-β2 mini- | 92 a.a. | C7V, C16A, K25R, | Residues |
| 7M2R-Del7-16 | | monomer with | | R26K, L51R, | 52-71 |
| (also referred to | | increased affinity for | | A74K, C77R, | |
| as “var1”) | | TβRII, reduced | | V79R, L89V, | |
| | | aggregation and | | I92V, K94R, | |
| | | improved folding | | T95K, I98V | |
| mmTGF-β2- | 7 | Human TGF-β2 mini- | 115 a.a. | K25R, R26K, | See |
| 7M-PRDC | | monomer including | | L89V, I92V, | FIG. 1D |
| | | Finger 1-2 and Finger | | K94R, T95K, I98V | |
| | | 3-4 grafted with cystine | | | |
| | | knot region of PRDC | | | |

As described in greater detail below, substitution of C77 with arginine, instead of serine as in mmTGF-β2-7M, together with substitution of V79 with arginine, enabled monomer formation and reduced aggregation of mmTGF-β2-7M2R (FIG. 1B). A reduction in misfolding was achieved by elimination of the C7-C16 disulfide by either C7V and C16A substitutions (mmTGF-β2-2M-Del7-16; FIG. 1C) or by replacement of the cystine knot region with that of PRDC (mmTGF-β2-7M-PRDC; FIG. 1D). Monomer mmTGF-β2-7M2R-Del7-16 (FIG. 1E) includes modifications to both reduce aggregation and improve folding.

SEQ ID NO: 1 - WT human TGF-β2
ALDAAYCFRNVQDNCCLRPLYIDFKRDLGWKWIHEPKGYNANFCAGACPY
LWSSDTQHSKVLSLYNTINPEASASPCCVSQDLEPLTILYYIGKTPKIEQ
LSNMIVKSCKCS
SEQ ID NO: 2 - mmTGF-β2
ALDAAYCFRNVQDNCCLRPLYIDFKRDLGWKWIHEPKGYNANFCAGACPY
RASKSPSCVSQDLEPLTILYYIGKTPKIEQLSNMIVKSCKCS
SEQ ID NO: 3 - mmTGF-β2-7M
ALDAAYCFRNVQDNCCLRPLYIDFRKDLGWKWIHEPKGYNANFCAGACPY
RASKSPSCVSQDLEPLTIVYYVGRKPKVEQLSNMIVKSCKCS
SEQ ID NO: 4 - mmTGF-β2-7M2R
ALDAAYCFRNVQDNCCLRPLYIDFRKDLGWKWIHEPKGYNANFCAGACPY
RASKSPRCRSQDLEPLTIVYYVGRKPKVEQLSNMIVKSCKCS
SEQ ID NO: 5 - mmTGF-β2-2M-Del7-16
ALDAAYVFRNVQDNCALRPLYIDFRRDLGWKWIHEPKGYNANFCAGACPY
RASKSPSCVSQDLEPLTILYYIGRTPKIEQLSNMIVKSCKCS
SEQ ID NO: 6 - mmTGF-β2-7M2R-Del7-16
ALDAAYVFRNVQDNCALRPLYIDFRKDLGWKWIHEPKGYNANFCAGACPY
RASKSPRCRSQDLEPLTIVYYVGRKPKVEQLSNMIVKSCKCS
SEQ ID NO: 7 - mmTGF-β2-7M-PRDC
KEVLASSQEALVVTERKYLKSDWCKLRPLYIDFRKDLGWKWIHEPKGYNA
NFCYGQCNSFYIPRHVKKEEDSFQSSAFCVSQDLEPLTIVYYVGRKPKVE
QLSNMIVKSCRCMSV

In some implementations, any of the above sequences include an N-terminal methionine (M) residue.

### Elimination or Reduction in the Propensity to Aggregate

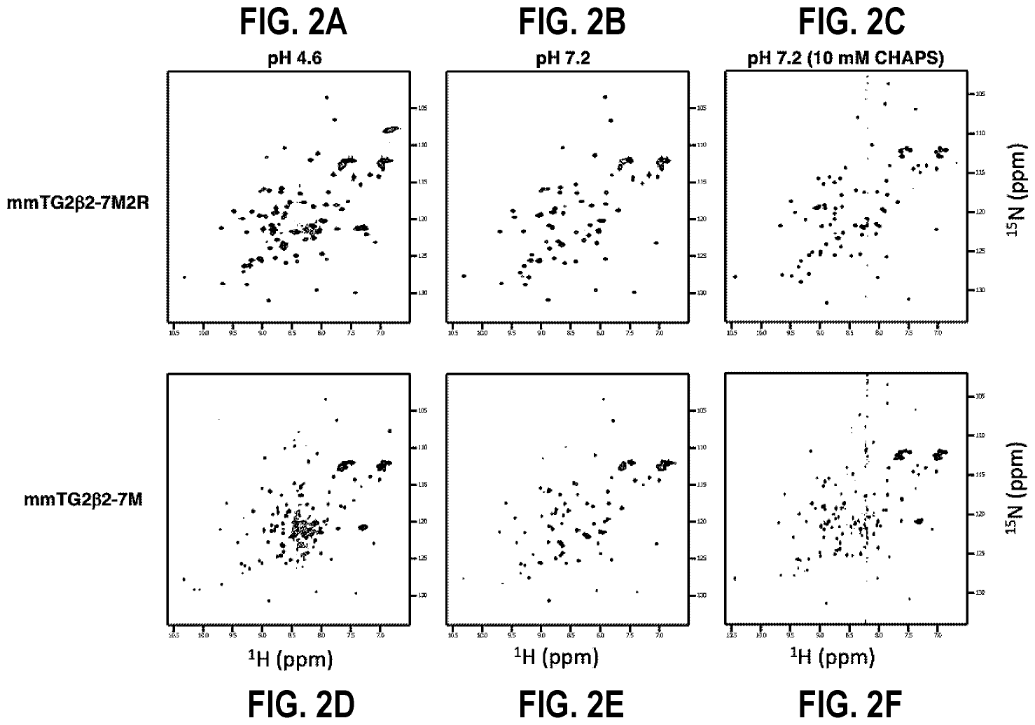

Modifications to eliminate or reduce the propensity of mmTGF-β2-7M to aggregate were first investigated. Though it was previously shown that the engineered mmTGF-β2-7M monomer is much less prone to aggregate than wild type TGF-β2, mmTGF-β2-7M nonetheless retains some propensity to aggregate (Kim et al., J Biol Chem 292(17):7173-7188, 2017). This was evident from the appearance of the amide backbone ¹H-¹⁵N signals, as detected by two-dimensional ¹H-¹⁵N NMR shift correlation (HSQC, heteronuclear single-quantum correlation) spectrum when recorded either in the absence of the non-denaturing detergent 3-[(3-Cholamidopropyl)dimethylammonio]-1-propanesulfonate (CHAPS) (FIGS. 2D, 2E) or in its presence (FIG. 2F). In the absence of CHAPS, the backbone amide signals were highly variable in intensity, with some barely detectable, particularly at pH 7.2 where the solubility of the protein is known to be reduced relative to that at pH 4.6. This type of variation in signal intensity is caused by the transient formation of higher order aggregates. The formation of such aggregates lengthens the rotational correlation time (τ_{c}) of the protein, thus broadening the NMR signals and causing the signal intensities to decrease. It has been observed that the addition of increasing concentration of CHAPS at either pH 4.6 or 7.2 leads to the improvement in the intensities of many signals and thus an increase in the uniformity of these in the observed spectrum (FIG. 2F). The improvement in signal intensities was dependent on the concentration of CHAPS, with substantial improvements occurring up to concentrations of about 10 mM.

It was hypothesized that the role CHAPS played in reducing the aggregation of mmTGF-β2-7M is due to transient formation of aggregates through some hydrophobic residues that remain in the region of the molecule, best described as the base of the fingers, which in the wild TGF-β2 homodimer were part of the dimer interface (FIG. 1A). Though several substitutions were tested that were found to have little effect on aggregate formation, substitution of two residues in mmTGF-β2-7M to arginine, S57R and V59R (FIG. 1B), which correspond to C77S and V79R substitutions with respect to the wild-type human TGF-β2 of SEQ ID NO: 1, significantly reduced the propensity to aggregate. This variant of mmTGF-β2-7M bearing the two resides replaced with arginine is designated as mmTGF-β2-7M2R (SEQ ID NO: 4). The evidence for reduced propensity to aggregate was the much more uniform NMR signal intensities that were observed for this variant, regardless of the pH or whether or not the non-denaturing CHAPS was added (FIGS. 2A-2C).

### Modifications to Improve Folding

TGF-β proteins are formed from monomers that are classified as having a cystine knot growth factor fold (CKGF) (Hinck et al., Cold Spring Harb Prospect Biol 8(12):a022103, 2016). This fold is present in all proteins of the TGF-β family, but is also found in many other signaling proteins and signaling protein antagonists in humans. These include the signaling proteins platelet-derived growth factor (PDGF), vascular-endothelial growth factor (VEGF), and nerve growth factor (NGF) and antagonists, such as noggin, sclerostin, and protein related to Dan and Cerubus (PRDC). The proteins of the TGF-β family are unique among CKGF proteins in that they all have an N-terminal pro-domain. Though the roles of the pro-domains are still being investigated, it is known that they have a regulatory role for many proteins of the family (Hinck et al., Cold Spring Harb Prospect Biol 8(12):a022103, 2016). This regulation comes about from binding of the pro-domains to the growth factor domain (GFD), sometimes with sufficient (nanomolar to sub-nanomolar) affinity to completely block the ability of the GFD to bind the type I and type II receptors. Some pro-domains, such as those for TGF-1, TGF-β2, and TGF-β3, not only bind the GFD with very high affinity, and thus maintain them in an inactive (latent) form until they are activated, but are also required for proper folding of the GFD. The GFDs of the TGF-βs, like that of other CKGF proteins, is characterized by a cystine knot, which is a structural motif stabilized by three disulfides (Schwarz, Biol Chem 398(12):1295-1308, 2017). The three disulfides are very close in space to one another and thus their formation is complex and there are many possible alternative topological arrangements in addition to the correct one.

This is relevant to mmTGF-β2-7M since it retains the cystine knot (as well as one additional disulfide, known as the 8-17 disulfide (corresponding to the cysteines at residues 7 and 16 relative to SEQ ID NO: 1, thus referred to herein as “7-16”). One way to produce mmTGF-β2-7M protein is to express it in bacteria in the form of insoluble inclusion bodies and refold the protein to form the native pairing of disulfides (Huang and Hinck, Methods Mol Biol 1344:63-92, 2016). The overall folding yields are nonetheless limited by aggregates that form as a result of mis-folding and improper pairing of its eight cysteine residues. mmTGF-β2-7M protein can also be produced by expressing the protein in a eukaryotic host as a secreted protein, but unlike wild type TGF-β homodimers, without a pro-domain. However, attempts at using this method for expression of mmTGF-β2-7M led to formation of significant misfolded disulfide linked aggregates.

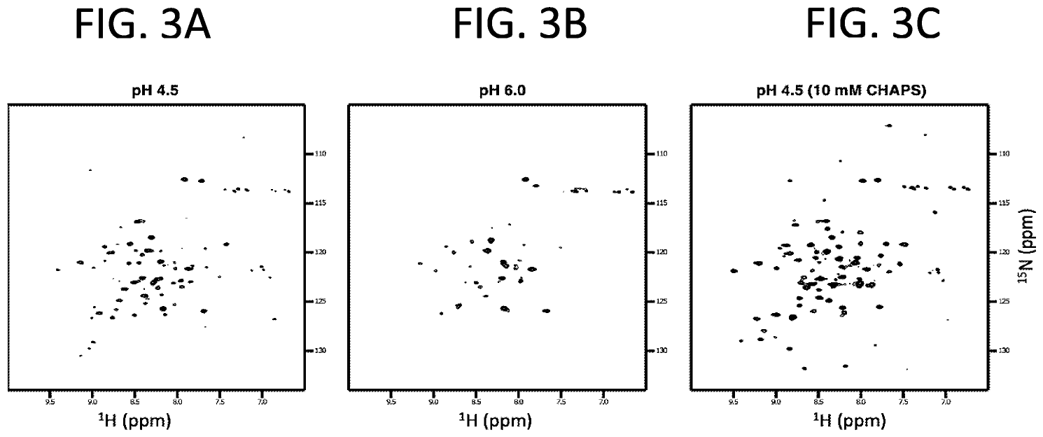

Thus, modifications aimed at improving the folding of mmTGF-β2-7M were investigated. To improve the folding, each of the four disulfides of mmTGF-β2-7M were eliminated, one disulfide at a time. To do this, the two cysteines that form each disulfide were substituted with a valine-alanine pair and then the modified protein was expressed, refolded, and purified according to previous procedures (Kim et al., J Biol Chem 292(17):7173-7188, 2017). To enhance the likelihood of attaining natively folded protein, the substitutions were generated in the context of the engineered TGF-β2 monomer, but with just two essential residues, K25 and K94, changed to those of TGF-β1, instead of seven substitutions as in mmTGF-β2-7M. These variants were still expected to bind TβRII with high affinity, but fold with improved efficiency since TGF-β2 is known to fold with much greater efficiency than TGF-β1 (Huang and Hinck, Methods Mol Biol 1344:63-92, 2016). The results showed that in this background, the variant with the cysteines that form the 7-16 disulfide substituted with valine and alanine, designed as mmTGF-β2-2M-Del7-16 (SEQ ID NO: 5; FIG. 1C) was natively folded (FIGS. 3A-3C), but the variants with the other three disulfides eliminated (15-78, 44-109 and 48-111) were non-native. There was notably significant variation in NMR signal intensities in the absence of CHAPS, suggestive of aggregation, though these disparities are lessened upon addition of CHAPS (FIGS. 3A-3C). The fact that the 7-16 disulfide can be eliminated without disrupting the folding of the protein indicates that this could lead to significant improvements in folding, whether the protein is produced in bacteria and refolded in vitro, or if the protein in produced in eukaryotic cells as a secreted protein.

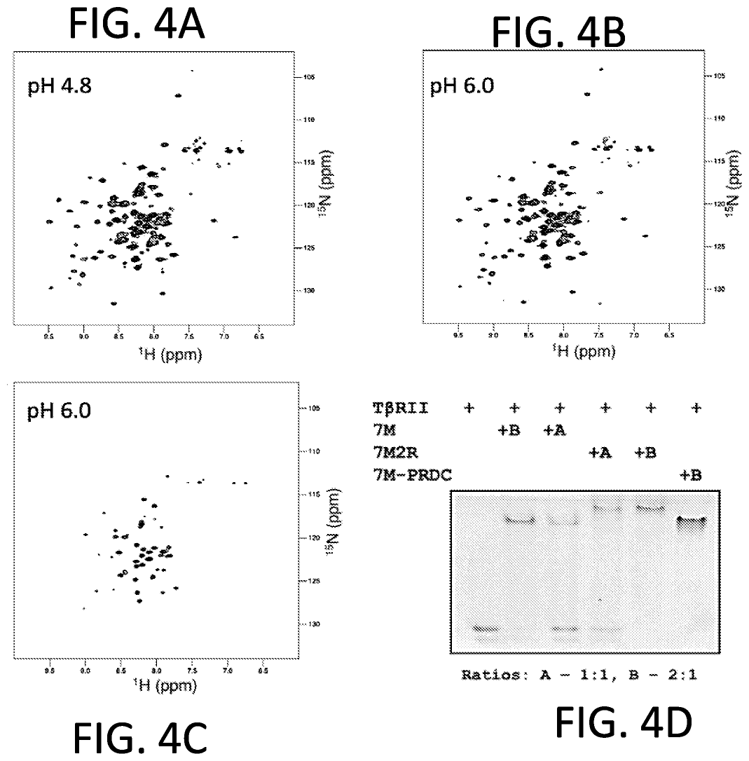

A third type of modification investigated was also aimed at improving the folding of mmTGF-β2-7M. The strategy chosen was to take advantage of the fact that there are some CKGF proteins, such as the bone morphogenetic protein (BMP) antagonist PRDC, that are produced naturally as monomers and do not have or rely upon a pro-domain for folding. To take advantage of the potential improvements in folding of PRDC, but to retain high affinity TβRII binding, a chimeric mmTGF-β2-7M:PRDC construct was generated in which the finger 1-2 and 3-4 regions of mmTGF-β2-7M, which are the regions responsible for binding TβRII, were grafted onto the cystine knot region of PRDC. This construct, designated as mmTGF-β2-7M-PRDC (SEQ ID NO: 7; FIG. 1D), was expressed in E. coli, refolded in a manner similar to that used for mmTGF-B27M (Kim et al., J Biol Chem 292(17):7173-7188, 2017), and purified to homogeneity using high-resolution cation exchange chromatography. Through NMR analysis, this protein was shown to be natively folded as evidenced by the dispersion of the amide signals well-outside of the random coil region, which corresponds to 7.9-8.5 ppm in the ¹H dimension (FIGS. 4A-4C). This indicates that the design was successful, with the cystine knot region of PRDC being well-integrated with the finger region of mmTGF-β2-7M.

### Binding Properties of mmTGF-β2-7M Variants

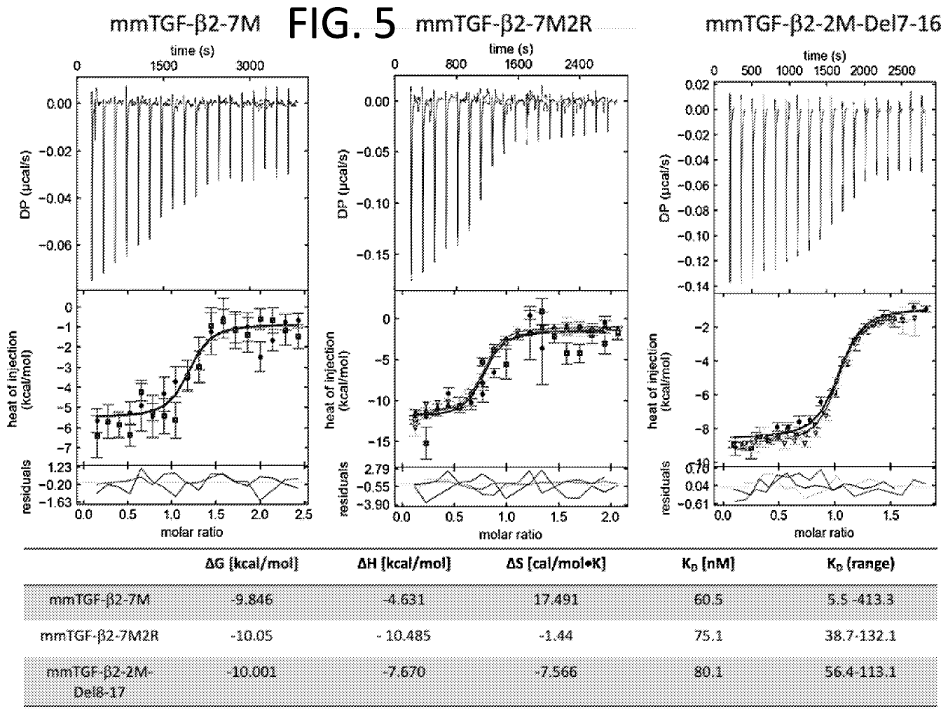

In order to be functional in cells and in vivo, a pre-requisite of any designed mmTGF-β2-7M variant is that it bind TβRII with high-affinity. In order to evaluate the ability of the mmTGF-β2-7M variants described herein (mmTGF-β2-7M2R (SEQ ID NO: 4), mmTGF-β2-2M-Del7-16 (SEQ ID NO: 5), and mmTGF-β2-7M-PRDC (SEQ ID NO: 7)) to bind TβRII, isothermal titration calorimetry (ITC) and native gels were used. The ITC binding experiments were performed by injecting increasing amounts of TβRII into mmTGF-β2-7M2R (SEQ ID NO: 4) or mmTGF-β2-2M-Del7-16 (SEQ ID NO: 5), with mmTGF-β2-7M (SEQ ID NO: 3) used a reference control. These titrations yielded readily detectable isotherms with a large negative enthalpy and a near 1:1 binding stoichiometry (FIG. 5). The fits of the integrated heats to a 1:1 binding model yielded disassociation constants (K_{D}^{S}) for binding TβRII of 75.1 nM and 80.1 nM for mmTGF-β2-7M2R and mmTGF-β2-2M-Del7-16 , respectively (FIG. 5). These K_{D}^{S} are within experimental error of that determined for mmTGF-β2-7M (60.5 nM) indicating that the substitutions introduced to reduce aggregation or improve folding had no deleterious effect on the ability of the protein to bind TβRII.

The binding of mmTGF-β2-7M-PRDC (SEQ ID NO: 7) was alternatively assessed using native gels. These do not provide a quantitative measurement of the K_{D}, though they are indicative of high affinity binding as detection of a complex requires that the two proteins remain bound on a timescale comparable to that of electrophoresis, which is on the order of an hour. The native gel showed that mmTGF-β2-7M, mmTGF-β2-7M2R, and mmTGF-β2-7M-PRDC all formed a band that migrates approximately one-fourth of the length of the gel, while TβRII runs over nearly the full-length of the gel (FIG. 4D). This, together with the previous finding that the mmTGF-β2-7M, mmTGF-β2-7M2R, and mmTGF-β2-7M-PRDC alone do not enter the gel, suggests that all three of these proteins bind TβRII with high affinity. This is consistent with the ITC results for the mmTGF-β2-7M and mmTGF-β2-7M2R variants, and indicates that this also true for mmTGF-β2-7M-PRDC.

### Inhibitory Properties of mmTGF-β2-7M Variants

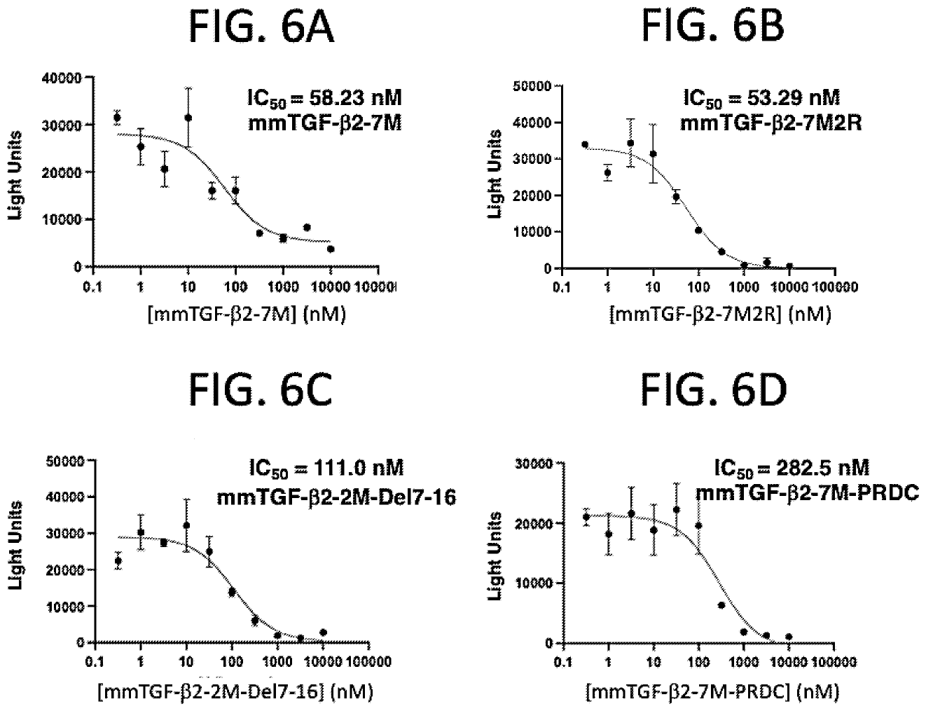

In order to be functional in vivo, any designed mmTGF-β2-7M variant should inhibit TGF-β signaling in cells. In order to assess this for the disclosed mmTGF-β2-7M variants, mmTGF-β2-7M2R (SEQ ID NO: 4), mmTGF-β2-2M-Del7-16 (SEQ ID NO: 5), and mmTGF-β2-7M-PRDC (SEQ ID NO: 7), a HEK-293 TGF-β luciferase reporter cell line in which the cells are stably transfected with a TGF-β CAGA enhancer element fused to a luciferase reporter gene was used. To assess inhibitory potential with this assay, the cells were plated in 96-well plates and varying concentrations of mmTGF-β2-7M2R, mmTGF-β2-2M-Del7-16, and mmTGF-β2-7M-PRDC were added, with mmTGF-β2-7M used as a control. After 30 minutes, TGF-β signaling was stimulated by adding TGF-β3 to a final concentration of 10 pM and after 12 hours, the cells were lysed and the luciferase activity was assessed. The results obtained showed that mmTGF-β2-7M2R, mmTGF-β2-2M-Del7-16 , and mmTGF-β2-7M-PRDC each potently inhibited signaling induced by TGF-β3, with fitted IC₅₀ values of 53 nM, 111 nM, and 283 nM, respectively (FIGS. 6B-6D). The values for mmTGF-β2-7M2R and mmTGF-β2-2M-Del7-16 were both within a factor of two of that measured for mmTGF-β2-7M (FIG. 6A), indicating that both of these proteins are nearly as effective as mmTGF-β2-7M (IC₅₀ 58 nM). While still potent, the IC₅₀ for mmTGF-β2-7M-PRDC is 283 nM, which is about 5-fold reduced relative to mmTGF-β2-7M. This indicates that although mmTGF-β2-7M-PRDC is a functional TGF-β inhibitor, its potency may be compromised slightly due to some small changes in the orientations of the two finger regions.

### SUMMARY

The mmTGF-β2-7M variants disclosed herein harbor substitutions that reduce their propensity to aggregate and increase their propensity to fold. The mmTGF-β2-7M variants were each shown to retain the ability to bind TβRII with high affinity and to potently inhibit TGF-β3 signaling in cultured cells. Therefore, the disclosed mmTGF-β2-7M variants possess attributes that improve their ability to be administered in vivo and thus provide new avenues for therapeutically intervening to attenuate TGF-β mediated disease progression.

In view of the many possible implementations to which the principles of the disclosed invention may be applied, it should be recognized that the illustrated implementations are only preferred examples of the invention and should not be taken as limiting the scope of the invention. Rather, the scope of the invention is defined by the following claims. We therefore claim as our invention all that comes within the scope and spirit of these claims.

1. A recombinant transforming growth factor (TGF)-β2 monomer, comprising:
a cysteine to serine substitution, or a cysteine to arginine substitution, at an amino acid residue corresponding to residue 77 of SEQ ID NO: 1;
a deletion of the α3 helix corresponding to amino acid residues 52-71 of SEQ ID NO: 1;
a lysine to arginine substitution at an amino acid residue corresponding to residue 25 of SEQ ID NO: 1;
a leucine to arginine substitution at an amino acid residue corresponding to residue 51 of SEQ ID NO: 1;
an alanine to lysine substitution at an amino acid residue corresponding to residue 74 of SEQ ID NO: 1;
a lysine to arginine substitution at an amino acid residue corresponding to residue 94 of SEQ ID NO: 1; and
(i) an arginine to lysine substitution at an amino acid residue corresponding to residue 26 of SEQ ID NO: 1; a valine to arginine substitution at an amino acid residue corresponding to residue 79 of SEQ ID NO: 1; a leucine to valine substitution at an amino acid residue corresponding to residue 89 of SEQ ID NO: 1; an isoleucine to valine substitution at an amino acid residue corresponding to residue 92 of SEQ ID NO: 1; a threonine to lysine substitution at an amino acid residue corresponding to residue 95 of SEQ ID NO: 1; and an isoleucine to valine substitution at an amino acid residue corresponding to residue 98 of SEQ ID NO: 1;
(ii) a cysteine to valine substitution at an amino acid residue corresponding to residue 7 of SEQ ID NO: 1; and a cysteine to alanine substitution at an amino acid residue corresponding to residue 16 of SEQ ID NO: 1; or
(iii) a cysteine to valine substitution at an amino acid residue corresponding to residue 7 of SEQ ID NO: 1; a cysteine to alanine substitution at an amino acid residue corresponding to residue 16 of SEQ ID NO: 1; an arginine to lysine substitution at an amino acid residue corresponding to residue 26 of SEQ ID NO: 1; a valine to arginine substitution at an amino acid residue corresponding to residue 79 of SEQ ID NO: 1; a leucine to valine substitution at an amino acid residue corresponding to residue 89 of SEQ ID NO: 1; an isoleucine to valine substitution at an amino acid residue corresponding to residue 92 of SEQ ID NO: 1; a threonine to lysine substitution at an amino acid residue corresponding to residue 95 of SEQ ID NO: 1; and an isoleucine to valine substitution at an amino acid residue corresponding to residue 98 of SEQ ID NO: 1.

2. The recombinant TGF-β2 monomer of claim 1, comprising a cysteine to arginine substitution at an amino acid residue corresponding to residue 77 of SEQ ID NO: 1.

3. The recombinant TGF-β2 monomer of claim 2, wherein the amino acid sequence of the TGF-β2 monomer comprises or consists of SEQ ID NO: 4 or SEQ ID NO: 6.

4. (canceled)

5. The recombinant TGF-β2 monomer of claim 1, comprising a cysteine to serine substitution at an amino acid residue corresponding to residue 77 of SEQ ID NO: 1.

6. The recombinant TGF-β2 monomer of claim 5, wherein the amino acid sequence of the TGF-β2 monomer comprises or consists of SEQ ID NO: 5.

7. A recombinant transforming growth factor (TGF)-β2 monomer, wherein the amino acid sequence of the TGF-β2 monomer comprises or consists of SEQ ID NO: 7.

8. The recombinant TGF-β2 monomer of claim 1, which is PEGylated, glycosylated or hyper-glycosylated.

9. (canceled)

10. The recombinant TGF-β2 monomer of claim 1, further comprising a radiotherapy agent, a cytotoxic agent for chemotherapy, a drug, an imaging agent, a fluorescent dye, or a fluorescent protein tag.

11. A fusion protein comprising the recombinant TGF-β2 monomer of claim 1 and a heterologous protein.

12. The fusion protein of claim 11, wherein the heterologous protein comprises a protein tag, an Fc domain, albumin, an albumin-binding polypeptide, an antibody, an antigen-binding fragment of an antibody or a targeting moiety.

13. The fusion protein of claim 11, wherein:
the fusion protein is a single-chain polypeptide;
the fusion protein forms a dimeric polypeptide;
the fusion protein is heterodimeric; or
the fusion protein is multimeric.

14-16. (canceled)

17. An isolated nucleic acid molecule encoding the recombinant TGF-β2 monomer of claim 1.

18. The nucleic acid molecule of claim 17 operably linked to a promoter.

19. A vector comprising the nucleic acid molecule of claim 18.

20. An isolated cell comprising the vector of claim 19.

21. (canceled)

22. A composition comprising:
the recombinant TGF-β2 monomer of claim 1; and
a pharmaceutically acceptable carrier, diluent, or excipient.

23. A method of inhibiting TGF-β signaling in a cell, comprising contacting the cell with an effective amount of the recombinant TGF-β2 monomer of claim 1.

24. A method of inhibiting TGF-β signaling in a subject having a disease or disorder associated with aberrant TGF-β signaling, comprising administering to the subject an effective amount of the recombinant TGF-β2 monomer of claim 1.

25. A method of treating a disease or disorder associated with aberrant TGF-β signaling in a subject, comprising administering to the subject a therapeutically effective amount of the recombinant TGF-β2 monomer of claim 1.

26. The method of claim 25, wherein:
the disease or disorder associated with aberrant TGF-β signaling is a fibrotic disorder;
the disease or disorder associated with aberrant TGF-β signaling is breast cancer, brain cancer, pancreatic cancer, prostate cancer, skin cancer, bladder cancer, liver cancer, ovarian cancer, renal cancer, endometrial cancer, colorectal cancer, gastric cancer, skin cancer or thyroid cancer;
wherein the disease or disorder associated with aberrant TGF-β signaling is an ocular disease; or
the disease or disorder associated with aberrant TGF-β signaling is a genetic disorder of connective tissue.

27-29. (canceled)
